(19) United States
(12) Patent Application Publication
(10) Pub. No.: US 2026/0335016 A1
(43) Pub. Date: Oct. 29, 2026

(54) PATIENT-SPECIFIC IMPLANT AND METHOD OF MANUFACTURING SAME

(71) Applicant: ChoSpine, LLC, Englewood Cliffs, NJ (US)

(72) Inventor: Samuel K. Cho, Englewood Cliffs, NJ (US)

(73) Assignee: ChoSpine, LLC, Englewood Cliffs, NJ (US)

(21) Appl. No.: 19/191,853

(22) Filed: Apr. 28, 2025

(51) Int. Cl. A61B 34/10; A61F 2/30; A61F 2/44; A61F 2/46; G16H 10/60

(52) U.S. Cl. CPC A61B 34/10; A61F 2/442; A61F 2/4455; G16H 10/60; A61B 2034/102; A61B 2034/105; A61B 2034/108; A61F 2002/30952; A61F 2002/4633

(57) ABSTRACT

A system and method for generating a patient-specific implant is described herein. The system includes a processor configured to receive, from an imaging device, patient-specific image data of a patient's joint; create a 3D model of the patient's joint based on the received patient-specific image data; obtain normative values of joint parameters for a joint type that matches the joint of the received patient-specific image data; modify the 3D model of the patient's joint based on the normative values of joint parameters for said joint type to create a normalized 3D model of the patient's joint; and modify a 3D model of a joint implant based on the normalized 3D model of the patient's joint.

### FIELD OF INVENTION

The subject disclosure concerns treatment of spinal or other bone abnormalities by the use of a patient-specific implant or prosthesis, and a method and system for designing such implant or prosthesis.

### BACKGROUND

Surgeons and clinical practitioners use imaging tools such as MRI, CT and X-rays to evaluate if a patient's spine or other bone structure is within a so-called healthy or normal configuration. If the patient's spine or other bone structure is outside of a healthy or normal configuration, e.g., a diseased configuration, the surgeon decides how much restoration or positional variance must be imparted to return the patient to a healthy or normal configuration, which often results in the pain experienced by the patient being reduced or even eliminated. Such restoration may be performed using procedures such as spinal fusion or joint replacement, e.g., disc replacement, interbody fusion or arthroplasty. Because patients are of different ages, gender, ethnicity, sizes, weights, and so on, the healthy or normal configuration of patients may differ from individual to individual.

### BRIEF SUMMARY

A system for generating a patient-specific implant comprising: a processor configured to: receive patient-specific image data of a patient's joint; generate a 3D model of the patient's joint based on the received patient-specific image data; obtain normative values of joint parameters for a joint type that matches the patient's joint of the received patient-specific image data; modify the 3D model of the patient's joint based on the normative values of the joint for the joint type to create a normalized 3D model of the patient's joint; and generate a 3D model of a joint implant based on the normalized 3D model of the patient's joint.

The 3D model of the joint implant is an interbody spinal implant, a knee implant, a hip implant, a shoulder implant, an ankle implant, an elbow implant, or a shoulder implant.

The patient-specific image data is pre-operative image data of the patient's joint. The patient-specific image data is intra-operative image data of the patient's joint after portions of the joint have been resected. The joint parameters include bone morphology, joint or intervertebral disc spacing, and/or bone size. The processor is further configured to modify the normalized 3D model of the patient's joint based on a virtual surgery of the joint. The virtual surgery of the joint comprises a virtual resection or milling of a portion of the joint of the normalized 3D model.

The processor is further configured to: receive patient-specific medical data of the patient; and modify the 3D model of the joint implant based on the received patient-specific medical data. The obtained normative values are obtained via machine learning techniques. The processor is further configured to: receive surgeon-specific data regarding a surgery for the patient's joint ; and modify the 3D model of the joint implant based on the received surgeon-specific data. The 3D model of the joint implant includes joint bones and/or fibrocartilage of the joint.

A patient-specific interbody spinal implant comprising: a surface morphology based on patient-specific image data of an intervertebral joint or disc for which the interbody spinal implant is intended to be implanted and normative values of said intervertebral joint or disc obtained via machine learning techniques. The surface morphology of the implant is further based on a surgeon's surgical preferences for implantation of the interbody implant.

A method of producing a patient-specific implant comprising: receiving patient-specific image data of a patient's joint; creating a 3D model of the patient's joint based on the received patient-specific image data; obtaining normative values of joint parameters for a joint type that matches the patient's joint of the received patient-specific image data; modifying the 3D model of the patient's joint based on the obtained normative values of the joint parameters for the joint type to create a normalized 3D model of the patient's joint; obtaining a 3D model of a joint implant suitable for implantation within the patient's joint; and modifying the 3D model of the joint implant based on the normalized 3D model of the patient's joint.

The method further comprising manufacturing a joint implant based on the modified 3D model of the joint implant. The 3D model of the joint implant is an interbody spinal implant, a knee implant, a hip implant, a shoulder implant, an ankle implant, an elbow implant, or a shoulder implant. The 3D model of the joint implant includes joint bones and/or fibrocartilage of the joint. The patient-specific image data is pre-operative image data of the patient's joint and/or intra-operative image data of the patient's joint after portions of the joint have been resected.

The method further comprises modifying the normalized 3D model of the patient's joint based on a virtual surgery of the joint. The method further comprises: receiving patient-specific medical data of the patient; modifying the 3D model of a joint implant based on the received patient-specific medical data; receiving surgeon-specific data regarding a surgery for the patient's joint; and modifying the 3D model of the joint implant based on the received surgeon-specific data.

### BRIEF DESCRIPTION OF THE VIEWS OF THE DRAWINGS

The foregoing summary, as well as the following detailed description of the exemplary embodiments of the subject disclosure, will be better understood when read in conjunction with the appended drawings. For the purpose of illustrating the present disclosure, there are shown on the drawings exemplary embodiments. It should be understood, however, that the subject application is not limited to the precise arrangements and instrumentalities shown.

FIG. 1 illustrates a lateral view of an example human spinal column;

FIG. 2A illustrates a superior view of an example human lumbar vertebra;

FIG. 2B illustrates a lateral view of two example vertebral bodies forming a functional spinal unit;

FIG. 3 illustrates a schematic diagram of a system for generating a patient-specific implant in accordance with an exemplary embodiment of the subject disclosure;

FIGS. 4A-4C illustrate aspects of an example cervical spine of a patient;

FIG. 5 illustrates an example coronal X-ray of a cervical spine of a patient;

FIG. 6A illustrates an example preoperative lateral X-ray of an example cervical spine depicting a collapsed disc;

FIGS. 6B and 6C illustrate example postoperative lateral X-rays of the example cervical spine of FIG. 6A depicting a restoration of the cervical spine;

FIG. 7 illustrates a schematic of a cross-sectional image of an example lumbar vertebra;

FIG. 8 illustrates a schematic of an example image depicting a Kambin's triangle;

FIG. 9A illustrates a preoperative X-ray of an example lateral lumbar spine having an intervertebral disc that is collapsed and flat;

FIG. 9B illustrates a postoperative X-ray of an example lateral lumbar spine demonstrating restoration of the height and segmental lordosis of the intervertebral disc following a transforaminal lumbar interbody fusion (TLIF);

FIG. 10 illustrates an example X-ray of a lumbar spine in which a patient-specific-implant is used;

FIG. 11A illustrates a schematic of an example patient-specific implant having static dimensions;

FIG. 11B illustrates a schematic of an example expandable patient-specific implant having dynamic dimensions;

FIG. 12A illustrates an example X-ray of a varus knee pre-operation;

FIG. 12B illustrates an example X-ray of the varus knee of FIG. 12A post-operation;

FIG. 13A illustrates an example X-ray of a valgus knee pre-operation;

FIG. 13B illustrates an example X-ray of the valgus knee of FIG. 13A post-operation; and

FIG. 14 illustrates a method of generating a patient-specific implant according to an exemplary embodiment as described herein.

### DETAILED DESCRIPTION

Reference will now be made in detail to the various exemplary embodiments of the subject disclosure illustrated in the accompanying drawings. Wherever possible, the same or like reference numbers will be used throughout the drawings to refer to the same or like features. It should be noted that the drawings are in simplified form and are not drawn to precise scale. Certain terminology is used in the following description for convenience only and is not limiting. Directional terms such as top, bottom, left, right, above, below and diagonal, are used with respect to the accompanying drawings. The term “distal” shall mean away from the center of a body. The term “proximal” shall mean closer towards the center of a body and/or away from the “distal” end. The words “inwardly” and “outwardly” refer to directions toward and away from, respectively, the geometric center of the identified element and designated parts thereof. Such directional terms used in conjunction with the following description of the drawings should not be construed to limit the scope of the subject disclosure in any manner not explicitly set forth. Additionally, the term “a,” as used in the specification, means “at least one.” The terminology includes the words above specifically mentioned, derivatives thereof, and words of similar import.

“About” as used herein when referring to a measurable value such as an amount, a temporal duration, and the like, is meant to encompass variations of ±20%, ±10%, ±5%, ±1%, or ±0.1% from the specified value, as such variations are appropriate.

“Substantially” as used herein shall mean considerable in extent, largely but not wholly that which is specified, or an appropriate variation therefrom as is acceptable within the field of art. “Exemplary” as used herein shall mean serving as an example.

Throughout this disclosure, various aspects of the subject disclosure can be presented in a range format. It should be understood that the description in range format is merely for convenience and brevity and should not be construed as an inflexible limitation on the scope of the subject disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Furthermore, the described features, advantages and characteristics of the exemplary embodiments of the subject disclosure may be combined in any suitable manner in one or more embodiments. One skilled in the relevant art will recognize, in light of the description herein, that the present disclosure can be practiced without one or more of the specific features or advantages of a particular exemplary embodiment.

In other instances, additional features and advantages may be recognized in certain embodiments that may not be present in all exemplary embodiments of the subject disclosure.

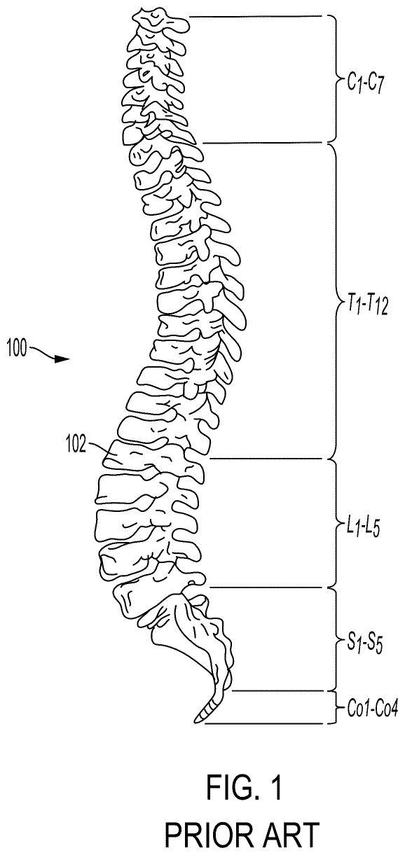

FIG. 1 depicts an example human spinal column 100. The spinal column 100 includes a series of thirty-three stacked vertebrae 102 divided into five regions. The cervical region includes seven vertebrae, known as C1-C7. The thoracic region includes twelve vertebrae, known as T1-T12. The lumbar region contains five vertebrae, known as L1-L5. The sacral region is comprised of five normally-fused vertebrae, known as S1-S5, while the coccygeal region contains four fused vertebrae, known as Co1-Co4.

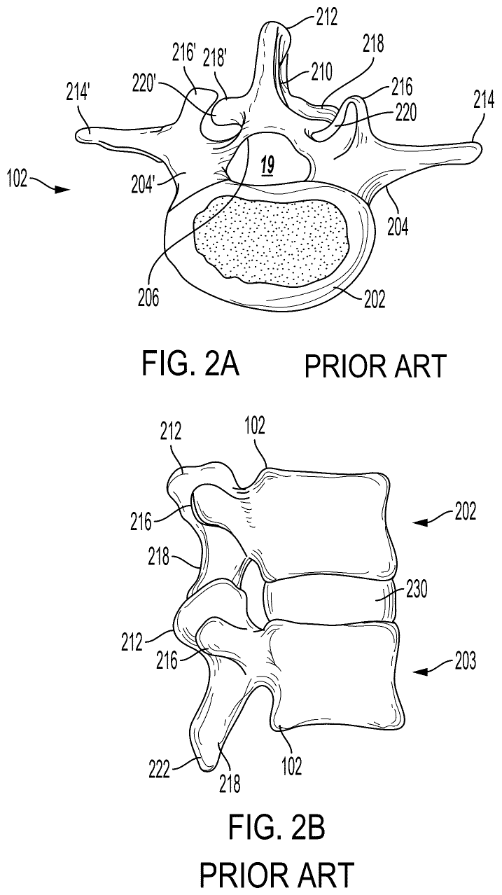

FIG. 2A depicts a superior plan view of an example human lumbar vertebra 102. The vertebra 102 includes a vertebral body 202. As best shown on FIG. 2A, two short bony protrusions, the pedicles 204, 204′, extend dorsally from each side of the vertebral body 202 to form a vertebral arch 206 which defines the vertebral foramen 208. At the posterior end of each pedicle 204, the vertebral arch 206 flares out into broad plates of bone known as the laminae 210. The laminae 210 fuse with each other to form a spinous process 212. The spinous process 212 provides for muscle and ligamentous attachment. A smooth transition from the pedicles 204 to the laminae 210 is interrupted by the formation of a series of processes. Two transverse processes 214, 214′ thrust out laterally, one on each side, from the junction of the pedicle 204 with the lamina 210.

The transverse processes 214, 214′ serve as levers for the attachment of muscles to the vertebrae 102. Four articular processes, two superior 216, 216′ and two inferior 218, 218′, also rise from the junctions of the pedicles 16 and the laminae 20. The superior articular processes 216, 216′ are sharp oval plates of bone rising upward on each side of the vertebrae, while the inferior processes 218, 218′ are oval plates of bone that jut downward on each side. FIG. 2B illustrates a lateral elevational view of two example vertebral bodies (202, 203) forming a functional spinal unit. As best shown on FIGS. 2A and 2B, the superior and inferior articular processes 216 and 218 each have a natural bony structure known as a facet or facet surface. The superior articular facet 220 faces medially upward, while the inferior articular facet 222 faces laterally downward. An intervertebral disc 230 between each adjacent vertebra 102 (with stacked vertebral bodies shown as 202, 203 in FIG. 2B) permits gliding movement between the vertebrae 102. The structure and alignment of the vertebrae 102 thus permit a range of movement of the vertebrae 102 relative to each other.

Injuries, diseases, inflammation, and other disorders of the joint may result in patients that may cause improper alignment and/or functioning of joints or surrounding areas. Examples of joints applicable to the subject disclosure that may encounter pathologies and disorders include, but not limited to, a patient's spine including vertebrae and intervertebral discs, knee joints, elbow joints, wrist joints, shoulder joints, and the like. Spinal pathologies and disorders may include scoliosis and other curvature abnormalities, kyphosis, degenerative disc disease, disc herniation, osteoporosis, spondylolisthesis, stenosis, tumor, and fracture. Disorders of the joint may result in symptoms including deformity, pain, nerve damage, and partial or complete loss of mobility. As used herein, a joint includes the bones, ligaments, tendons, fibrocartilage and the various other components or portions of the joint.

A system and method is described herein to restore a proper shape and/or alignment to an injured, diseased, or inflamed joint of a patient. Such system and method can include designing and/or generating (e.g., manufacturing) a patient-specific implant. The system and method described herein can restore the shape and/or alignment of the diseased joint to that of a patient having a healthy joint. In exemplary embodiments the system and method can perform a procedure or surgical procedure on one or more portions or surrounding portions of a joint of a patient. The surgical procedure may include performing a resection upon the joint or a portion of the joint e.g., a bone or intervertebral disc, a milling of the joint or a portion of the joint, and the like.

Examples of joints in which the exemplary embodiment of the subject disclosure are applicable to include the spine including vertebrae and intervertebral discs, knee joints including bones, ligaments, tendons and meniscus, elbow joints, wrist joints, shoulder joints, hip joints, ankle joints, and the like, of the patient. There may be many benefits for restoring a diseased joint and/or disc to that of a patient having a healthy joint, as described herein. For example, restoring a diseased intervertebral disc to that of a patient having a healthy intervertebral disc can decompress neural elements near the discs, may allow a surgeon to gain surgical access and visualization to the disc space, may allow an implant (e.g., patient-specific implant) to better fit, and the like. Implants applicable to the subject disclosure can include interbody implants, spacers, fusion cages, artificial disc replacements, total joints including knee implants, hip implants, ankle implants, shoulder implants, wrist implants and the like.

Interbody implants are configured to be placed in a space between two bones, such as two vertebrae. For example, an interbody implant is a spacer that can be placed in a space between two vertebrae to promote bone healing and/or fusion. In fusion surgeries, an intervertebral disc can be surgically removed prior to the placement of the interbody implant. In such embodiments the lower (inferior) side of an interbody implant can be configured to abut at least a portion of an upper (superior) side of a first vertebrae and the superior endplate of the interbody implant can abut at least a portion of an inferior endplate of a second vertebrae. Patient-specific interbody implants can be designed and/or generated for optimal fit in the space created by removal of the disc and adjustment of the relative position of vertebrae. In embodiments the design and/or generation of the patient-specific implant can be performed, using digital tools. In other embodiments the design of the implant can be automated, such as via machine learning and artificial intelligence techniques.

In an exemplary embodiment, the shape of the patient-specific implant can be based on a combination of the patient's preoperative (e.g., injured, diseased, inflamed) shape and the shape of an individual or target population of healthy individuals having a normative (e.g., healthy) shape. The shape of the implant can allow the structure of the patient to match or substantially match a shape of a joint having a normative function or structure. For example, when replacing and/or supplementing a joint or intervertebral disc of a patient with a patient-specific implant described herein, the joint and/or disc of the patient is expected to restore or substantially restore the motion or stability of the joint or intervertebral disc of a patient to a normative and/or healthy state.

As described herein, normative values for joints can be provided. The normative values can relate to joint parameters that include bone and/or intervertebral disc morphology, joint or intervertebral disc spacing, bone size (e.g., height, length), joint or intervertebral disc angulation, ligament size and shape, ligament balancing, and the like. The normative values can be obtained via images of joints and/or discs of individuals e.g., a target group of healthy individuals, other than the patient that include normative functioning and/or structure. In an exemplary embodiment the normative values relate to individuals other than the patient that have similar characteristics or parameters as the patient. Such characteristics can include physical characteristics, such as a similar height, weight, age, sex, activity level, bone density, etc., as the patient. For example, characteristics can include normative values of individuals who play tennis having a similar age and weight of the patient. In an embodiment the characteristics of individuals having similar characteristics as the patient can be provided via machine learning techniques.

Machine learning techniques can be used to determine the size and configuration (e.g., the optimal size and configuration) of the implant to be used for the patient. The size and/or configuration of the implant can be based on the space available within a body for a healthy person. For example, a machine learning model can be trained using data and/or parameters for joints of healthy persons. In such an example, an aggregate of data of healthy individuals can include patient attributes and values, such as height of the patient, age of the patient, sex of the patient. Data of healthy persons can include joint health attributes and values, such as very healthy, healthy, not healthy joint. Data can further include joint physical attributes and values, such as distance between two bones of a joint, curvature of the joint, size of joint bones, ligaments, tendons, fibrocartilage. e.g., intervertebral disc, and the like.

The machine learning model can be trained to determine relationships and corresponding attributes useful for designing and/or generating a patient-specific implant, such as healthy individual attributes, healthy joint and/or disc health attributes, healthy joint and/or disc physical attributes, and so on. Based on the patient information and information from other healthy individuals, the machine learning model can provide joint attributes of a joint having a desired health attribute based on individuals having attributes similar to the patient having surgery. An implant configuration can be designed and/or generated based on the joint attributes of the joint having a desired health attribute of individual(s) having attributes similar to the patient having surgery.

In exemplary embodiments, the joint of a patient may be modified so that the joint represents a size and/or configuration of a normative joint. For example, the joint of a patient may be modified (e.g., resected or milled) to restore the shape and/or alignment of the joint from a diseased state to a normative (e.g., healthy) state. The modification of the joint may be virtual or physical and can cause the joint of the patient to match the characteristics of an individual having a normative joint characteristics. In an embodiment in which a virtual modification of the joint is performed, a patient anatomy can be virtually modified prior to surgery. Such virtual modification can be performed to allow the surgeon to formulate a plan for how the actual surgery should be performed.

A patient-specific implant can be generated using patient-specific image data of a patient's joint. The patient-specific image data of a patient's joint can be generated via an imaging device. A 3D model of the patient's joint or intervertebral disc can be created based on the patient-specific image data. Normative values of joint parameters can be obtained for a joint type that matches the joint or intervertebral disc of the received patient-specific image data. The 3D model of the patient's joint can be modeled based on the normative values of joint parameters for said joint type to create a normalized 3D model of the patient's joint. A 3D model of a joint implant can be generated based on the normalized 3D model of the patient's joint.

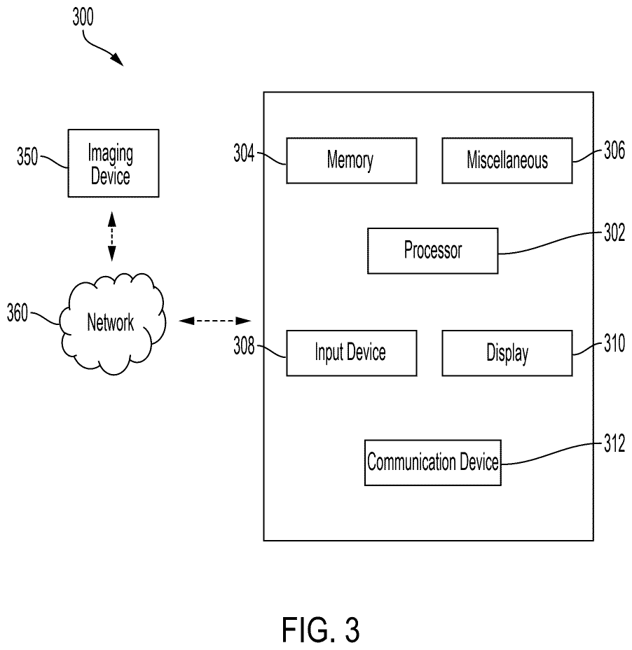

FIG. 3 illustrates an exemplary system 300 of the subject disclosure for designing and/or generating a patient-specific implant. The system 300 can include one or more of a memory 304, processor 302, input device 308, display 310, communication device 312, imaging device 350, and network 360. The system 300 can include other devices, such as those described as miscellaneous 306. One or more of the devices of system 300 can be found within a single device, although in other embodiments one or more devices of system 300 can be distributed across multiple devices.

The processor 302 may be a CPU, GPU, HPU, and the like. The processors 302 can be a single processing unit or multiple processing units in a device or distributed across multiple devices. The processor 302 can be coupled to other hardware devices, for example, with the use of a bus, such as a PCI bus or SCSI bus. Input devices 308 can include, for example, a mouse, a keyboard, a touchscreen, an infrared sensor, a touchpad, a wearable input device, a camera-or image-based input device, a microphone, or other user input devices. The processor 302 and memory 304 can be in operative communication with one another e.g., such that the processor can execute computer instructions stored within the memory.

Memory 304 can include one or more of various hardware devices for volatile and non-volatile storage, and can include both read-only and writable memory. For example, the memory 304 can comprise random access memory (RAM), various caches, CPU registers, read-only memory (ROM), and writable non-volatile memory, such as flash memory, hard drives, floppy disks, CDs, DVDs, magnetic storage devices, tape drives, device buffers, and so forth. The memory 304 can include program memory that stores programs and software, such as an operating system, computer instructions, and application programs. As described herein, memory 304 can receive and store one or more of data relating to a patient, other than a patient, a surgeon, and the like.

The system 300 can include a display 310 that can be used to display text, models, virtual procedures, surgical plans, implants, graphics, and/or images. In some embodiments, display 310 can provides graphical and textual visual feedback to a user. In some implementations, the display 310 can includes one or more devices, such as the input device 308, such as when the input device 308 is a touchscreen or is equipped with an eye direction monitoring system. In some embodiments, display 310 can be separate from the input device 308. Examples of display devices include an LCD display screen, an LED display screen, a projected, holographic, or augmented reality display (such as a heads-up display device or a head-mounted device), and so on.

In some implementations, the system 300 can include a communication device 312, such as a network device, which may enable system to communicate with one or more external devices, such as imaging device 350. Communication device 312 may enable system 300 to communicate with one or more external devices wirelessly or via a wire. The communication device 312 can communicate with an external device via network 360. In embodiments, network 360 can be the Internet, although imaging device 350 can communicate images of the patient to system via any known wired or wireless communication protocol. The system 300 can receive information relating to a patient via communication device 312. For example, images of the patient may be transmitted from imaging device 350 to communication device 312 of the system 300 via network 360.

The system 300 may include one or more miscellaneous devices 306, such as a network card, video card, audio card, USB, firewire or other external device, camera, printer, speakers, CD-ROM drive, DVD drive, disk drive, or Blu-Ray device. Other miscellaneous devices 306 can also include input ports for information from directly connected medical equipment such as imaging apparatuses, including MRI machines, X-ray machines, CT machines, etc.

As described herein, patient data may be received via the communication device 312. The patient data can include image data (e.g., medical image data) obtained via one or more imaging techniques that can include, without limitation, CT scans (e.g., 3D CT scans, CMCT scans, etc.), X-ray images, MRI images, or other imaging data. The image data can include structural information (e.g., bone structure, structure of fibrocartilage e.g., of an intervertebral disc or meniscus, etc.) of the patient, or the like. In an embodiment, the image data can include pre-operative information of a patient, such as pre-operative images of a joint of the patient. The image data can include intra-operative images of the joint of the patient. The pre-operative information can include joints of the patient that are injured, diseased, inflamed, and the like.

The intra-operative images of the joint of the patient can include images of the joint e.g., of the joint bones and fibrocartilage of the joint being modified (e.g., being resected or milled) and/or after bones of the joint or fibrocartilage e.g., an intervertebral disc) have been modified. The system 300 can generate one or more images of a patient via the image device 350. Example imaging devices 350 may include one or more X-ray Machines, CT (computed tomography) Scanners, MRI Machines, Ultrasound Machines, although such listing of machines is for illustration purposes and is non limiting. The imaging devices 350 can generate one or more he images that may include MRI scans of a spine, CT scans, digital images, X-rays, and the like.

Information relating to the patient may be stored in memory 304. For example, the memory 304 can store images of the patient (e.g., pre-operative images of the patient, intra-operative images of the patient, post-operative images of the patient). The images of the patient may include images of patient's joints in a diseased state. The memory 304 can store information describing characteristics of the patient, such as the sex, weight, height, etc. of the patient. In exemplary embodiments, information relating to individuals other than the patient may be stored by the memory 304. For example, the memory 304 can store images of people other than the patient, information relating to the patient or people other than the patient (e.g., sex, weight, height, etc. of people other than the patient), and so on. The memory 304 can store additional information, such as information relating to preferences (e.g., surgical preferences) of one or more surgeons, and the like. For example, the memory 304 can store whether a particular surgeon prefers to resect portions of a vertebrae or intervertebral disc, prefers not to resect e.g., an intervertebral disc, and so on.

The system 300, via the processor 302, can create/generate a model (e.g., a 3D model) of the structure of a patient's joint (such as the spine of the patient) based on patient information (e.g., image data of the patient). In an exemplary embodiment, the processor 302 can obtain and/or receive normative values e.g., of the vertebrae and intervertebral parameters for the spine, that match the fibrocartilage joint type structure of the patient. The 3D model of the patient's structure (e.g., joint) can be modified based on the obtained and/or received normative values. In an example, attributes of the 3D model of the patient's structure (e.g., joint) can be interpolated or substantially matched to be similar to the normalized 3D model of the patient's joint.

As described herein, the normative values and/or 3D model can be based on joints of individuals other than patient, and may be based on those individuals having an anatomy that is similar to the patient. For example, the normative values can be based on one or more healthy joint belonging to individuals having medical attributes similar or substantially the same as the patient and/or of similar age, sex, height, etc., as the patient. The processor 302 may be configured to modify the 3D model of the patient's structure (e.g., joint) based on the normative values to create a normalized 3D model of the patient's structure (e.g., joint). Based on the normalized 3D model of the patient's structure, a 3D model of a joint implant may be generated and/or modified. A joint implant can be manufactured based on the generated 3D model of the joint implant.

The system 300 can analyze, via the processor 302, patient data to identify or develop a virtual model (e.g., 3D model) of one or more portions of the patient, such as one or more joints including all portions or portions of the joint, of the patient. The processor 302 can use image data from one or more other individuals other than the patient to identify or develop a virtual model (e.g., 3D model) that is a normalized representation of the subject joint. The system 300 may allow a medical practitioner (e.g., surgeon) to modify the one or more of the virtual models (e.g., 3D model of the patient's joint, normalized 3D model of the patient's joint, etc.) by performing a virtual procedure upon the portion of the patient, such as to perform a virtual resection of the joint (e.g., vertebra, intervertebral disc, knee joint, etc.), a milling of the joint, and so on. The 3D model may represent the joint in one or more of a pre-operative state, an intra-operative state, a normalized state, and the like. The 3D model may represent the joint or intervertebral disc in a diseased state or normalized state, along with one or more states between a diseased state and a normalized state.

As described herein, the system 300 can obtain normative values of the patient's structure, such as anatomic features of the structures in a healthy or normalized (e.g., correct or healthy) state. In such examples the system 300 can provide options for procedures (e.g., virtual procedures, actual procedures) that can result in the structure transforming from a diseased state to a normalized state. In embodiments a desired joint (e.g., a normalized joint bone or intervertebral disc, a joint bone or intervertebral disc suggested by the system, and/or a joint based on preferences of a surgeon) may be overlayed upon the joint of the patient (e.g., a diseased joint of the patient). Having such overlay of the normalized joint upon the diseased joint of the patient allows a surgeon/user to perform a virtual procedure by following the example of the normalized joint. The anatomical features of the normalized joint can include features of individuals that include similar medical data as the patient and/or other information as the patient, such as individuals having the same sex, age, health rating, height, weight, or other attributes as the patient. In an exemplary embodiment a 3D model of the patient and/or individual other than the patient can include the spine including intervertebral disc, cervical vertebrae, thoracic vertebrae, and lumbar vertebrae. In other embodiments the 3D model can include other joints, such as the knees, elbows, wrists, ankles, shoulders, and the like.

In embodiments, the system 300 can use machine learning techniques to determine how the joint of the patient (e.g., diseased joint of the patient) should be modified to be reflective of a joint in a normalized state. For example, processor 302 can use machine learning techniques to determine how e.g., the spine (including vertebrae and intervertebral discs), of a patient should be modified (e.g., resected, milled, etc.) to be reflective of a normative (e.g., healthy, or non-diseased) spine. In exemplary embodiments, the machine learning techniques may use information relating to individuals other than patient having similar features/parameters as the patient, such as individuals or population group having a similar age, height, sex, weight, etc., as the patient.

For example, a neural network model can be trained to determine or identify normative joint parameters that are similar to the diseased joint of a subject patient. The neural network can be trained via information relating to the patient, information relating to individuals other than the patient, information relating to surgeon preferences, and so on. The information used to train the neural network can include medical images or medical imaging (e.g., camera images, still images, scans, MRI scans, CT scans, X-ray images) of the patient as well as medical images of individuals other than the patient, description information relating to the patient (such as sex, height, weight, etc.), description information relating to individuals other than the patient, and so on. This neural network can receive the information relating to the patient, individuals other than the patient and the surgeon and output information relating to normative joint data.

Upon normative values being obtained of joint parameters for a joint type that matches the joint of the received patent-specific image data, the 3D model of the patient's joint may be modified based on the normative values of such joint or parameters. A 3D model of a joint implant may be generated and/or modified based on the normalized 3D model of the patient's joint. The system 300 can design and/or generate a patient specific implant based on the 3D model of the joint implant. The 3D model of the joint implant may be representative of the patient's joint modified based on normative joint data. For example, upon the system 300 determining how the joint of the patient can be modified by, based on, or to represent a normative joint, the processor 302 can determine an implant that can be best fit, or matched, or be used based on the modified joint of the patient.

For example, patient-specific interbody implants can be designed and/or generated for optimal fit with the modified joint of the patient. Such optimal fit can be used to place the vertebrae and/or discs within a healthy configuration of the patient. In an exemplary embodiment, the patient-specific interbody implant can be used to convert vertebrae or intervertebral disc configuration within a patient from a diseased configuration to a healthy configuration. The patient-specific interbody implant can be used to convert vertebrae or intervertebral disc configuration to a healthy configuration by placing the interbody implant within a negative space created by removal of a disc and adjustment of the relative position of vertebrae.

As described herein, the size and/or shape of the patient-specific implant may be determined prior to the surgery using preoperative imaging and software techniques allowing the surgeon to perform virtual procedures, such as virtual resecting and/or milling of one or more joints (e.g., spine, knees, elbows, et.) of the patient. For example, the patient-specific interbody implants can be designed and/or generated based on normative joint data of a subject joint. The size and the shape of the patient-specific implant may be provided to the surgeon or other user via display 310.

In an exemplary embodiment, the patient-specific implant (e.g., the patient-specific implant designed and/or generated by the processor 302) can be immobile. For example, the patient-specific implant can be fused to one or more bones of the patient, such as fixed to the cranial and caudal bones of the patient. In other embodiments, the patient-specific implant may include a mobile mechanism such as artificial disc replacement that can be used in interbody surgeries. In an embodiment the implant may involve two or levels of the spine, such as those used in corpectomy cages.

In embodiments the patient-specific implant can relate to one or more areas of the spine, such as one or more of cervical, thoracic, and lumbar areas of the spine. The patient-specific implant can match (e.g., partially match) the mammalian intervertebral disc anatomy of the patient. For example, the patient-specific implant can match (e.g., partially match) the mammalian intervertebral disc anatomy of the patient upon the processor 302 determining how the mammalian intervertebral disc anatomy should be altered to represent a normative joint or intervertebral disc. In an exemplary embodiment the patient-specific implant can match (e.g., partially match) the mammalian intervertebral disc anatomy of the patient upon the mammalian intervertebral disc anatomy being altered (e.g. virtually or physically) to represent a normative joint or intervertebral disc.

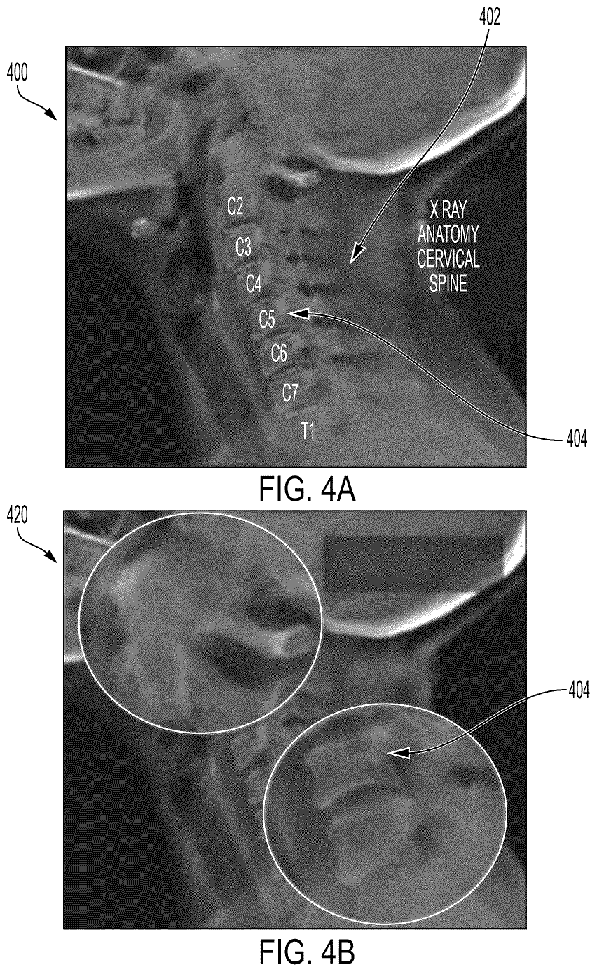

FIGS. 4A-4C show X-rays 400, 420, 450 depicting different aspects of an example cervical spine 402 of a patient. FIG. 4A shows the seven vertebrae (C1-C7) of the cervical spine 402 including vertebra 404, as well as T1 of the thoracic region. FIG. 4B shows an exploded vertebra 404 of cervical region C5, as shown on FIG. 4A. FIG. 4C shows areas 410 within the exploded portion of the cervical region C5 including vertebra 404 of FIG. 4B that may be modified (e.g., resected, milled) based on a determination of the processor 302, as described herein.

In an exemplary embodiment, the processor 302 may receive one or more images of a patient's joint. As provided in FIG. 4A, the images of the patient may be of the patient in a preoperative (e.g., diseased) state. The processor 302 can determine areas, such as one or more joints (e.g., vertebrae and intervertebral disc) within a patient that should be modified. The processor 302 can determine areas within the patient that should be modified based on one or more parameters. For example, as described herein, the processor 302 can determine that vertebra 404 of cervical region C5 of the patient should be modified (e.g., resected, milled) so that cervical region C5 of the cervical spine of the patient is representative of or substantially matches that of normative values of the cervical spine. In an exemplary embodiment, the normative values of the spine may be based on healthy cervical spines of individuals having a similar height, weight, sex, age, and like of the patient. In other embodiments, the normative values of the joint may be based on joints of healthy individuals without regard to whether they have similar characteristics as the patient.

The anterior and/or posterior aspect of the vertebra (e.g. cranial vertebra, cervical vertebra) of the patient may be modified (e.g., resected) to gain access to the intervertebral disc space and/or to adequately decompress the involved neural elements (spinal cord and nerve root(s)). For example, FIG. 4C shows an example lateral X-ray of a vertebra 404 of a patient with the anterior and posterior lip of the cranial endplate shaded 410 to demonstrate what should be resected during surgery. In such embodiment, the anterior lip is resected so that the surgeon can obtain an improved visual of the disc space, and the posterior lip may be resected for decompression of the spinal cord and nerve roots. The processor 302 can determine a configuration (e.g., size, shape, etc.) of a patient-specific implant that can fit (e.g., snugly fit) with the modified vertebra 404. In an exemplary embodiment the implant may be permanently fit (e.g., fused) with the modified vertebra 404.

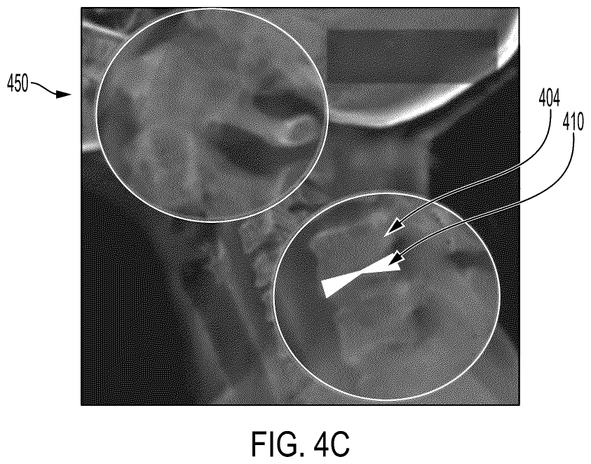

FIG. 5 shows an example coronal X-ray 500 of a cervical spine 502 of a patient. Dashed circles 504A, 504B are shown denoting uncovertebral joints that may be modified (e.g., resected, milled) to decompress the exiting nerve roots. In an embodiment, one or more of the uncovertebral joints may be modified to gain access to the intervertebral disc space and/or to adequately decompress the involved neural elements (spinal cord and nerve root(s)). The processor 302 can determine which joints or intervertebral discs of the patient are to be modified, and where the joints or intervertebral discs should be modified, based on normative joints or intervertebral discs. The normative joints or intervertebral discs may relate to individuals other than the patient that have one or more healthy joints or intervertebral discs.

For example, the processor 302 can receive the image 500 of a patient in which one or more uncovertebral joints of the patient are unhealthy. The processor 302 can receive normative data relating to individuals other than the patient. The normative data may relate to healthy joints and may include image data of the individual. In embodiments the normative data may relate to individuals that have similar characteristics as the patient, such as a similar height, weight, age, sex, and so on. The processor 302 can determine a modification of the joint of the patient such that after the modification of the joint results in the joint having a similar configuration as the normative joint or intervertebral disc of the individual. As described herein, the processor 302 can determine a configuration (e.g., size, shape) of an implant that corresponds to the joint of the patient in the joint's modified state. The processor 302 can determine the configuration of a patient-specific implant such that the implant fits (e.g., snugly fits) with the modified joint or portions of the joint. In an exemplary embodiment the implant may be permanently fit (e.g., fused) with the modified joint or modified portions of the joint.

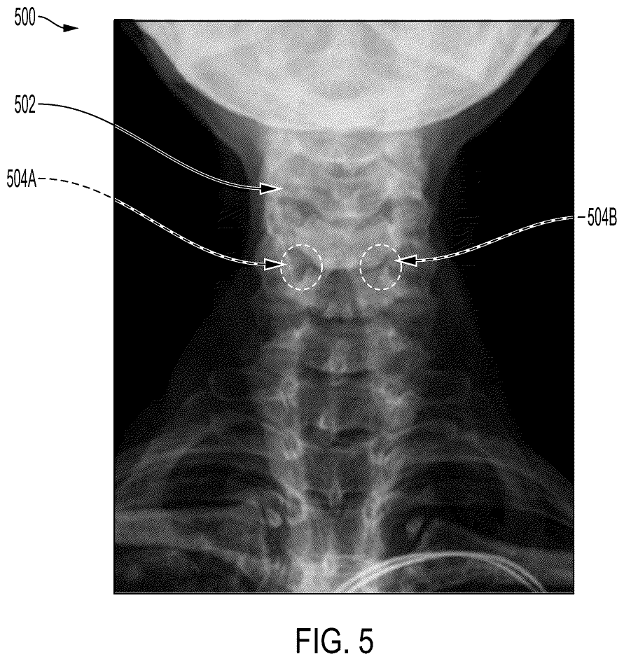

FIG. 6A shows a preoperative lateral X-ray 600 of an example cervical spine depicting a collapsed disc 602 with loss of proper height and lordosis. FIGS. 6B and 6C show postoperative lateral X-rays 650, 660 of the example cervical spine depicting a restoration of the cervical spine. In particular, FIGS. 6B, 6C show postoperative lateral X-rays 650, 660 of an example cervical spine in which device 610 restores the cervical spine 600 to a desired disc height and angulation. The restoration shown on FIGS. 6B, 6C can be performed according to the segmental lordosis, although in other embodiments the restoration can be performed upon the entire cervical lordosis spanning the second vertebra (C2) to the seventh vertebra (C7). The disc height and angulation (segmental lordosis) of the cervical spine 600 can be modified (e.g., surgically modified) to restore proper dimensions appropriate to the spine.

The processor 302 can determine the height and/or angulation in which the disc 602 may need to change based on normative discs and/or joints. The normative discs and/or joints may relate to individuals other than the patient that have one or more healthy joints and/or discs. For example, the processor 302 can receive the image 600 of a patient in which one or more discs of the patient are unhealthy. The processor 302 can receive normative data relating to individuals other than the patient. The normative data may relate to healthy discs and/or joints and may include image data of the individual. In embodiments the normative data may relate to individuals that have similar characteristics as the patient, such as a similar height, weight, age, sex, and so on.

The processor 302 can determine a modification of the joint of the patient such that, after the modification of the joint, the joint has a similar configuration as the normative values of the joint. In an embodiment the modification of the joint can include a device (e.g., device 610) placing the joint in a height and/or angulation to achieve or match normative values of the joint. The processor 302 can determine the configuration of a patient-specific implant such that the implant fits (e.g., snugly fits) within the joint e.g., the spine, to permit the spine to have a height and/or angulation similar to normative values of the spine. In an exemplary embodiment the implant may be permanently fit (e.g., fused) with a modified vertebra.

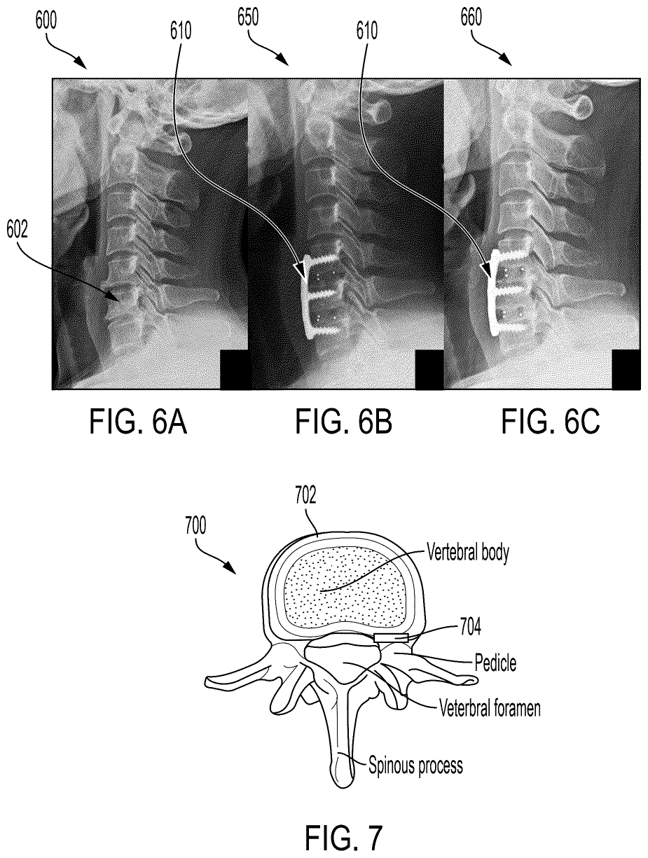

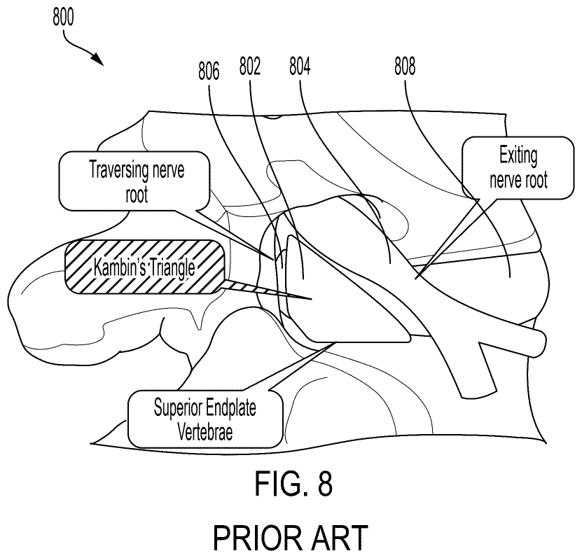

FIG. 7 shows a cross-sectional image 700 of an example lumbar vertebra. The lumbar vertebra includes an apophyseal ring 702, which is a dense and slightly protruding bone. In embodiments one or more portions of the lumbar vertebra, such as the apophyseal ring 702, can be modified (e.g., resected, milled, crushed). The portions can be modified for many reasons, such as for visibility and/or access. For example, one or more portions (e.g., portion 704) of the lumbar vertebra may be modified to gain access to the intervertebral disc space and/or to adequately decompress the involved neural elements (spinal cord and nerve root(s)). FIG. 8 shows an image 800 depicting an example Kambin's triangle 802. The Kambin's triangle 802 is a narrow surgical corridor bordered by an exiting nerve root 804, traversing nerve root 806, and the caudal pedicle. The disc 808 may be modified (e.g., resected, milled). After the disc 808 is modified, a small portion of the apophyseal ring 702 (FIG. 7) may be crushed or removed. In embodiments the apophyseal ring 702 (FIG. 7) may be crushed or removed to even the apophyseal ring 702 to the level of the endplate for better visualization, removal of the disc 808, and/or cage insertion. The processor 302 can determine which portions of the patient (e.g., apophyseal ring 702 (FIG. 7), disc 808 (FIG. 8)) should be modified, and where the portions should be modified, based on normative joints.

For example, the processor 302 can determine that the apophyseal ring 702 (FIG. 7) and/or disc 808 of a patient should be modified, and where the apophyseal ring 702 and/or disc 808 should be modified, based on normative joint bones or intervertebral discs. The normative joint bones or intervertebral discs may relate to individuals other than the patient that have one or more healthy joint bones or intervertebral discs. For example, the processor 302 can receive the image 700 of a patient in which one or more vertebrae and/or discs of the patient are unhealthy. The processor 302 can receive normative data relating to individuals other than the patient. The normative data may relate to healthy vertebrae and/or discs and may include image data of the individual. In embodiments the normative data may relate to individuals that have similar characteristics as the patient, such as a similar height, weight, age, sex, and so on. The processor 302 can determine a modification of the joint (e.g., apophyseal ring 702) of the patient such that, after the modification, the joint has a similar configuration as the normative joint values. As described herein, the processor 302 can determine a configuration (e.g., size, shape) of an implant that corresponds to the joint of the patient in the joint's modified state. The processor 302 can determine the configuration of a patient-specific implant such that the implant fits (e.g., snugly fits) with the modified joint. In an exemplary embodiment the implant may be permanently fit (e.g., fused) with the modified joint bone.

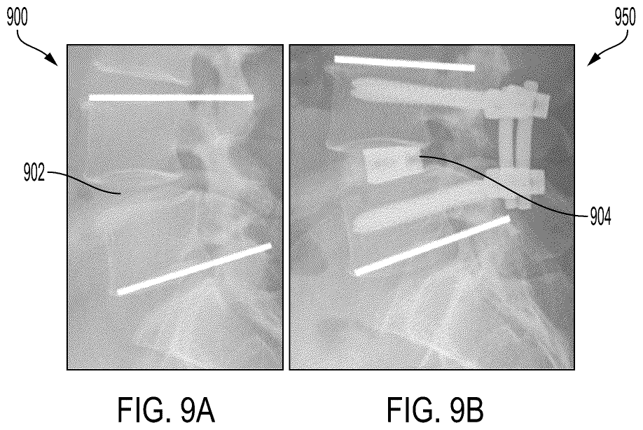

FIG. 9A shows an example preoperative X-ray 900 of an example lateral lumbar spine having a disc 902 that is collapsed and flat. FIG. 9B shows a postoperative X-ray 950 of an example lateral lumbar spine demonstrating restoration of the height and segmental lordosis of the disc 902 following a transforaminal lumbar interbody fusion (TLIF). Although FIG. 9B shows a transforaminal lumbar interbody fusion, a posterior or transforaminal lumbar interbody fusion may be performed upon the lumbar spine. In embodiments in which a posterior or transforaminal lumbar interbody fusion is performed, a portion of the cranial and caudal apophyseal ring may be modified (e.g., resected, milled, crushed). For example, as described herein, the apophyseal ring (e.g., cranial and caudal apophyseal ring) may be modified to gain access to the intervertebral disc space through the surgical corridor within the Kambin's triangle (FIG. 8). With regard to the lumbar spine, when performing a posterior or transforaminal lumbar interbody fusion using one or more devices (e.g., patient-specific implant 904), the height and/or angulation (segmental lordosis) of the disc may be changed to a height and/or angulation (segmental lordosis) of a normative disc.

The processor 302 can determine the height and/or angulation in which the disc 902 may need to change based on normative discs and/or joints. The normative discs and/or joints may relate to individuals other than the patient that have one or more healthy joints and/or discs. For example, the processor 302 can receive the image 900 of a patient in which one or more discs of the patient are unhealthy. The processor 302 can receive normative data relating to individuals other than the patient. The normative data may relate to healthy discs and/or joints and may include image data of the individual. In embodiments the normative data may relate to individuals that have similar characteristics as the patient, such as a similar height, weight, age, sex, and so on.

The processor 302 can determine a modification of the disc and/or joint of the patient such that, after the modification of the joint, the discs and/or joint have a similar configuration as the normative discs and/or joint of the individual. In an embodiment the modification of the disc and/or joint can include a device (e.g., device 904) placing the discs and/or joint in a height and/or angulation as normative discs and/or joints. The processor 302 can determine the configuration of a patient-specific implant such that the implant fits (e.g., snugly fits) within the spine to permit the discs and/or joint to have a height and/or angulation similar to normative discs. In an exemplary embodiment the implant may be permanently fit (e.g., fused) with the modified vertebra.

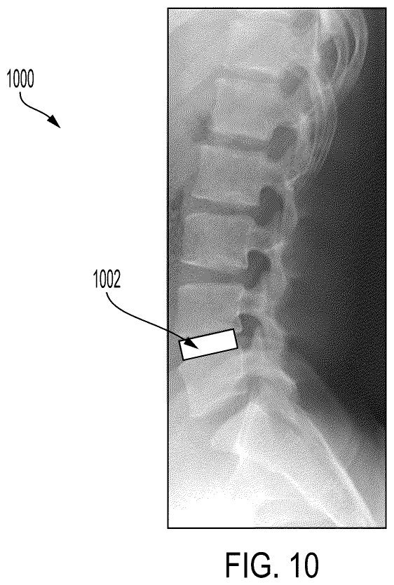

FIG. 10 shows an example X-ray 1000 of a lumbar spine in which a patient-specific implant 1002 (e.g., patient-specific cage, such as TLIF cage) is used. In the lumbar spine 1000, for the purposes of performing a posterior or transforaminal lumbar interbody fusion, the device 1002 may have one or more constant or standard dimensions (e.g., lengths, widths) while other devices include one or more dimensions that match or is modified based on the target disc of the patient. In an embodiment, because of the narrow corridor through the Kambin's triangle, the width of the device 1002 may include a range of 8-14 mm, including 9, 10, 11, 12 and 13 mm, with an exemplary width of 10-12 mm, including 10.5, 11, and 11.5 mm.

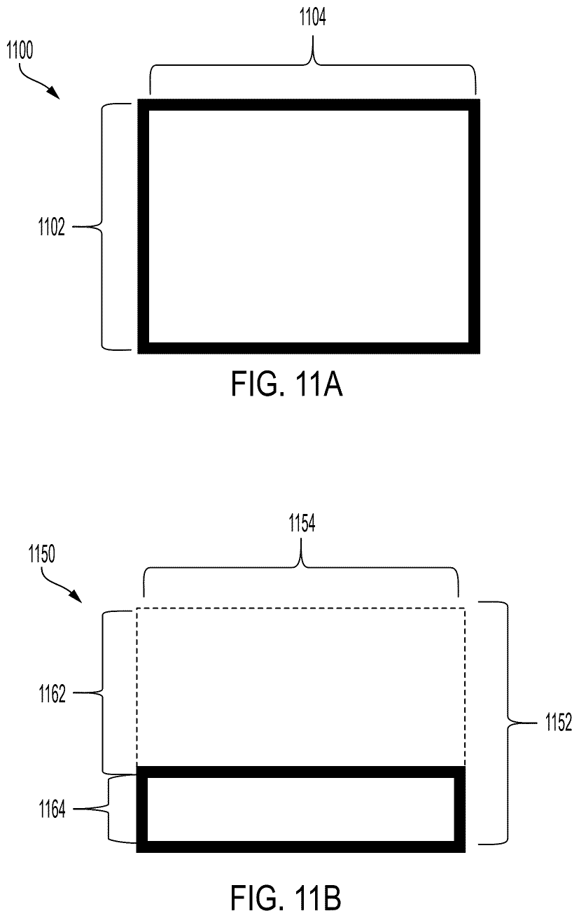

In an embodiment, a patient-specific implant can include sizes and/or configurations that are static (e.g., designed and/or generated with a static height, angulation, and/or width), such as the example patient-specific implant 1100 shown on FIG. 11A having a static height 1102 and width 1104. In other embodiments, the patient-specific implant can be designed and/or generated so that the sizes and/or configurations of the implant are dynamic (e.g., designed and/or generated with a dynamic height, angulation, and/or width), such as the patient-specific implant 1150 shown on FIG. 11B having a dynamic height 1152. As shown on FIG. 11B, expandable patient-specific implant 1150 has a collapsed height 1164. The height of the expandable patient-specific implant 1150 can be incrementally expandable according to expandable height 1162. In such embodiments the maximum height of the expandable patient-specific implant 1150 is the height 1152. Although FIG. 11B shows the expandable patient-specific implant 1150 expanding in height, in other embodiments the expandable patient-specific implant 1150 can expand or reconfigure in one or more areas, such as in height, width, depth, angle, and the like.

The expandable patient-specific implant 1150 can collapse to height 1164 so that the implant 1150 can enter a disc space that is smaller in dimension than patient-specific implant 1100 having height 1102. Once in the disc space, a surgeon can manipulate (e.g., via a knob) the expandable patient-specific implant 1150 so the implant expands. Collapsing the expandable patient-specific implant 1150 prior to insertion within a body of a patient allows the implant to fit within narrow surgical corridors, which can mitigate or remove a possibility of injuring neighboring tissues such as nerves while inserting the patient-specific implant. In an example, a surgeon may perform a TLIF using a collapsable patient-specific implant, such as expandable patient-specific implant 1150. In an embodiment, the implant can have a collapsed size of 7 mm disc height and 0 degree lordosis and can expand up to a 14 mm disc height and 20 degrees of lordosis. In such an example, a patient may require an implant having a 12 mm disc height and 15 degrees of lordosis. The expandable patient-specific implant 1150 can be manipulated (e.g., via a knob, gauge, etc.) to expand the implant to the desired dimensions.

The processor 302 can determine the size and/or configuration (e.g., height, width, and/or angulation) in which the expandable patient-specific implant 1150 may need to change based on normative values for the discs and/or joint bones. The normative joints may relate to individuals other than the patient that have one or more healthy joints and/or discs. For example, the processor 302 can receive an image of a patient in which one or more discs of the patient are unhealthy. The processor 302 can receive normative data relating to individuals other than the patient. The normative data may relate to healthy discs and/or joints and may include image data of the individual. The processor 302 can determine a modification of the disc and/or joint of the patient such that, after the modification of the joint, the discs and/or joint have a similar configuration as the normative discs and/or joint of the individual. In an embodiment the modification of the disc and/or joint can include a device placing the discs and/or joint in a height and/or angulation as normative discs and/or joints. The processor 302 can determine the configuration of a patient-specific implant such that the implant fits (e.g., snugly fits) within the spine to permit the discs and/or joint to have a height and/or angulation similar to normative discs. In an exemplary embodiment the implant may be permanently fit (e.g., fused) with the modified vertebra. In the above example, the processor can determine that the patient-specific implant should expand to a height of 12 mm disc and a lordosis of 15 degrees.

When modifying (e.g., resecting, milling) a joint and/or disc of a patient, it may be desired that the entirety of the joint and/or disc not be modified. For example, modifying an entire disc may result in damaging structures near the disc. As a result, in embodiments the patient-specific implant device (e.g., cage) as described herein may cover only a portion of the disc space of a patient. The length of the patient-specific implant device may be shorter (e.g., slightly shorter) than the entire anterior-to-posterior distance of the disc of the patient. The length of the patient-specific implant device may not extend beyond the disc, which may ensure the cage does not extend into a space where nerves or blood vessels surrounding the discs are located. As a result, having the length of the patient-specific implant device being shorter than the anterior-to-posterior distance of the disc (e.g., not extending beyond the disc) may prevent the patient-specific implant device from disturbing nerves or blood vessels surrounding the discs of the patient which may mitigate or eliminate the patient incurring resulting nerve or blood vessel problems. Thus, with respect to the lumbar spine and a posterior or transforaminal interbody fusion, the patient-specific implant may be undersized in one or more dimensions so as to mitigate or eliminate the patient incurring resulting nerve or blood vessel problems. The patient-specific implant may be undersized in one or more dimensions so that the implant can achieve a proper (e.g., snug) fit while not extending beyond the disc, which is advantageous as once the implant is installed it typically cannot be removed without potential harm to the patient.

With respect to the lumbar spine, when performing an anterior lumbar interbody fusion (ALIF) or transforaminal lumbar interbody fusion (TLIF), a portion of cranial and caudal apophyseal ring may be altered (e.g., resected). For example, a portion of the cranial and caudal apophyseal ring may be altered to gain access to the intervertebral disc space through the surgical corridor. In exemplary embodiments, ALIF and TLIF procedures may be performed in a similar manner, although the surgical corridor in an ALIF may be wider and may be bordered and limited by the great blood vessels. With respect to the lumbar spine, when performing an ALIF and/or TLIF, the disc height and angulation (segmental lordosis) may need to change in the surgically treated pathologic disc to restore proper dimensions appropriate for that level. The processor 302 can determine the disc height and angulation (segmental lordosis) in which the patient-specific implant may need to change based on normative discs and/or joints, as described herein. The normative discs and/or joints may relate to individuals other than the patient that have one or more healthy joints and/or discs.

When performing a lateral (trans-psoas) lumbar interbody fusion (LLIF), oblique (ante-psoas) lumbar interbody fusion (OLIF), or TLIF, a portion of the cranial and caudal apophyseal ring may be altered (e.g., resected) to gain access to the intervertebral disc space through the surgical corridor. When performing a LLIF, OLIF, and/or TLIF, the disc height and angulation (segmental lordosis) may be altered in the surgically treated pathologic disc. Such alteration of the surgically treated pathologic disc may be intended to restore desired dimensions of the disc that may be appropriate for the particular level of the disc. The processor 302 can determine the desired dimensions of the disc that may be appropriate for the particular level of the disc based on normative discs and/or joints, as described herein. The normative discs and/or joints may relate to individuals other than the patient that have one or more healthy joints and/or discs.

When performing a LLIF or OLIF, the patient-specific implant can be designed and/or generated to have one or more constant dimensions (e.g., widths). The patient-specific implant can have other dimensions (e.g., lengths, angulations) that may be configured to match the disc of the patient. The limitation of performing a LLIF and OLIF can include the great blood vessels anteriorly and the lumbar nerve roots posteriorly. In an example, the width of the patient-specific implant can be within a range of 10-20 mm, with an exemplary width of 14-18 mm. As described herein, the implant can have varying lengths, angulations, and heights and can be undersized in one or more dimensions so that the implant can achieve a proper (e.g., snug) fit while not extending beyond the disc. The processor 302 can determine the dimensions of the implant for performing a LLIF or OLIF based on normative discs and/or joints, as described herein.

The patient-specific implant can be used to replace one or more mobile joints or intervertebral discs. The patient-specific implant can be used to fully replace (e.g., total joint replacement) or partially replace (e.g., partial joint replacement) one or more mobile joints or intervertebral discs. In an exemplary embodiment, the patient-specific implant can be configured to match the joint or intervertebral disc anatomy. Examples of such joints include a hip, knee, shoulder, elbow, wrist, ankle, finger, and toe of a mammal. As described herein, the implant can be immobile, such as being fixed to proximal and distal bones of a patient. In other embodiments, the implant may include a mobile mechanism such as those used in artificial disc replacement surgeries.

In an embodiment a diseased bone of a patient may need to be altered (e.g., resected) to properly restore appropriate anatomy and/or function to the joint or intervertebral disc. In such embodiments, the patient-specific implant can be configured to partially match the joint or intervertebral disc anatomy. The implant can be configured to match the joint or intervertebral disc anatomy after the joint or intervertebral disc anatomy has been altered (e.g., resected, milled) to meet the needs of an intended medical procedure.

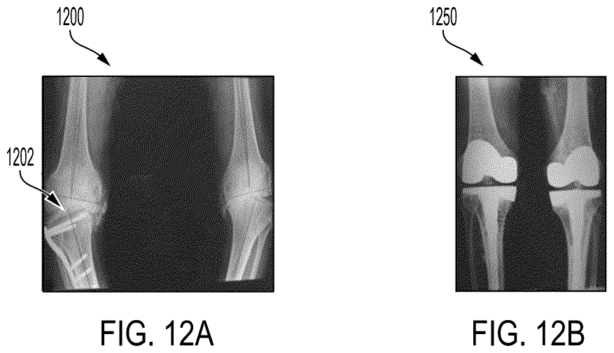

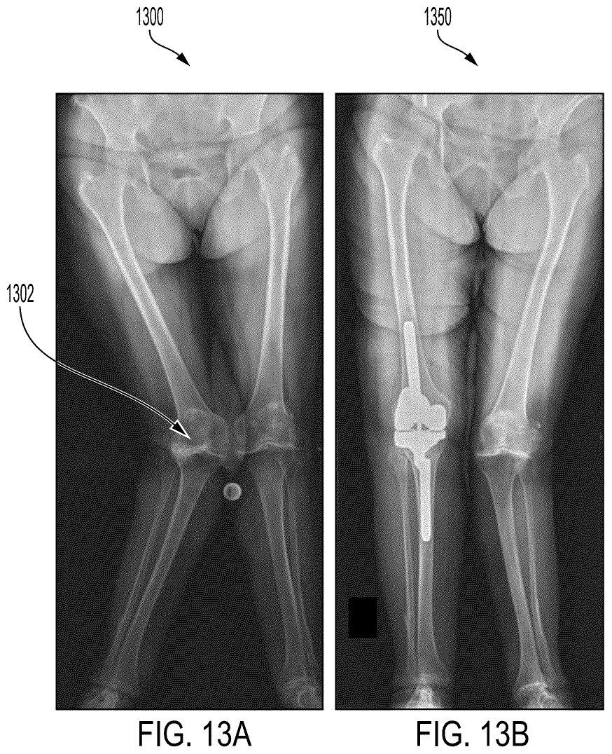

FIG. 12A shows an example X-ray 1200 of a varus knee pre-operation and FIG. 12B shows an example X-ray 1250 of a varus knee post-operation. FIG. 13A shows an example X-ray 1300 of a valgus knee pre-operation and FIG. 13B shows an example X-ray 1350 of a valgus knee post-operation. During joint (e.g., knee) replacement surgery, a diseased joint, such as knee joint 1202 and/or knee joint 1302, may be lengthened or shortened to properly balance the joint to restore appropriate anatomy and/or function. As described herein, patient data can be analyzed to design and/or generate a patient-specific implant, surgical plan, surgical instrument, or the like. The patient data can be analyzed to design and/or generate (e.g., manufacture) a patient-specific implant that conforms to a healthy joint or bone configuration of a patient. For example, the processor 302 can determine a modification of the knee joint of the patient such that, after the modification of the knee joint, the knee joint has a similar configuration as a normative joint of another individual. In an embodiment the modification of the joint can include a modification (e.g., resection, milling) of the knee joint to balance the joint to restore the knee joint to a normative knee joint. The processor 302 can determine the configuration of a patient-specific implant such that the implant fits (e.g., snugly fits) within the knee joint to permit the joint to have a height, angulation, and/or balance similar to normative knee joints. In an exemplary embodiment the implant may be permanently fit (e.g., fused) with the modified knee joint.

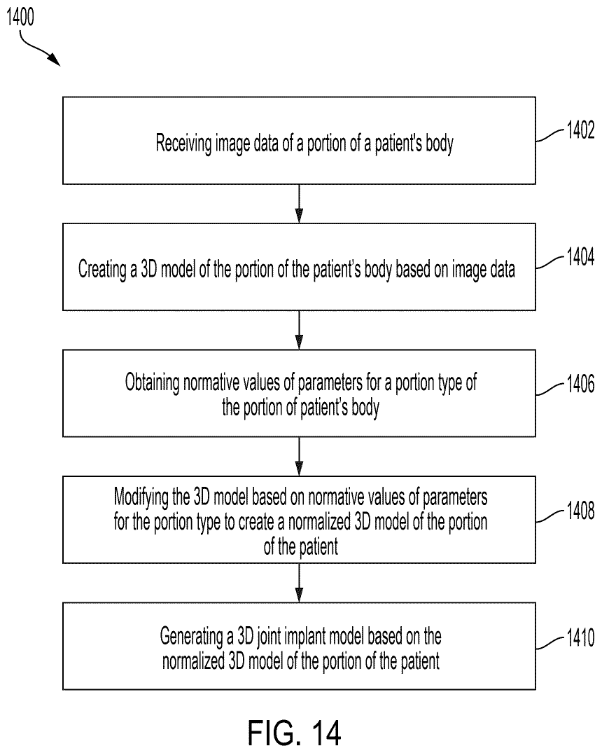

FIG. 14 shows an example method 1400 of designing and/or generating a patient-specific implant according to an exemplary embodiment of the subject disclosure. At Step 1402, data of a patient is received. The data can include medical image data obtained via one or more imaging techniques that can include, without limitation, CT scans (e.g., 3D CT scans, CMCT scans, etc.), X-ray images, MRI images, or other imaging data. The medical image data can be produced via imaging devices that may include one or more X-ray Machines, CT (computed tomography) Scanners, MRI Machines, Ultrasound Machines, although such listing of machines is for illustration purposes and is non limiting. The imaging devices can generate one or more images that may include MRI scans of a spine, CT scans, digital images, X-rays, and the like. The imaging data can include information of a patient such as structure information (e.g., joint structure, disc structure, etc.), or the like. The medical image data can relate to one or more anatomical portions of the patient, such as a joint including bones, ligaments, tendons and fibrocartilage and the like, of a patient. Joints of the patient may include the spine including vertebrae and intervertebral discs, knee joints, elbow joints, and the like of the patient. The medical image data can include one or more anatomical portions of the patient in a diseased state.

At Step 1404, a model (e.g., 3D model) of one or more portions of the patient is created based on the image data obtained in Step 1402. In exemplary embodiments, the model of the one or more portions of the patient may be created via software, such as anatomical software known in the art. The one or more portions of the patient may include one or more joints bones, ligaments, tendons and fibrocartilage, and the like, of a patient. At 1406, normative values of a portion type of the portion of the patient are obtained. The normative values of the portion of the patient include features of the portion in a healthy or corrected state. The normative values of the portion of the patient can be derived from individuals that have similar medical data as the patient and/or have similar characteristics as the patient, such as individuals having the same sex, age, health rating, height, weight, or other attributes of the patient.

In Step 1408, the 3D model of the portion of the patient is modified based on normative values, such as the normative values found in Step 1406. In an exemplary embodiment the model of the patient can be modified via virtual procedures that result in the portion of the patient in a diseased state becoming a normative state. For example, modifying the model of the portion of the patient based on the normative values can be used to create a normalized model (e.g., normalized 3D model) of the patient's joint or intervertebral disc. Procedures can be performed on the model of the patient to create the normalized model of the patient's joint or intervertebral disc. Such procedures can include virtual resections of the portion of the patient, virtual millings of the portion, and the like. The procedures can be performed iteratively, such as virtual iterative resections of the joint or intervertebral disc, virtual iterative millings of the joint or intervertebral disc, and the like. In embodiments a normative portion (e.g., joint) can be overlayed upon the portion of the patient (e.g., a diseased joint of the patient). Having such overlay of the normative portion upon the diseased portion of the patient can allow a medical practitioner to perform a modification of the patient (e.g., virtual or physical modification of the patient) by following the example of the normative portion.

In embodiments, machine learning techniques can be used to determine how the portion of the patient (e.g., diseased joint of the patient) should be modified to be reflective of a joint or intervertebral disc in a normative state. For example, machine learning techniques can be used to determine how a vertebrae of a patient should be modified (e.g., resected, milled, etc.) to be reflective of a normative (e.g., healthy, or non-diseased) vertebrae. In exemplary embodiments, the machine learning techniques may use information relating to individuals other than patient having similar features as patient, such as individuals having a similar age, height, sex, weight, etc., as patient.

For example, a neural network model can be trained to determine normative joints or intervertebral discs that are similar to the diseased joint or intervertebral disc of the patient. The neural network can be trained via information relating to the patient, information relating to individuals other than the patient, information relating to surgeon preferences, and so on. The information used to train the neural network can include images (e.g., camera images, still images, scans, MRI scans, CT scans, X-ray images) of the patient as well as image of individuals other than the patient, description information relating to the patient (such as sex, height, weight, etc.), description information relating to individuals other than the patient, and so on. This neural network can receive the information relating to the patient, individuals other than the patient and the surgeon and output information relating to the normative joint or intervertebral disc.

At 1410, a patient specific implant can be generated and/or designed based on the determined normative 3D model of the portion of the patient e.g., joint. For example, upon determining how the portion of the patient can be modified to represent a normative portion, an implant can be designed and/or generated that can be best used with the modified portion of the patient. Accordingly, patient-specific implants can be designed and/or generated for optimal fit with the modified portion of the patient. Such optimal fit can be used to place the bones within a configuration to substantially match that of a healthy individual.

In an exemplary embodiment, the patient-specific implant e.g., interbody, can be used to convert a bone or intervertebral disc configuration within a patient from a diseased configuration to a healthy configuration. The patient-specific interbody implant can be used to convert a bone or intervertebral disc configuration to a healthy configuration by placing the interbody implant within a negative space created by removal of a disc and adjustment of the relative position of vertebrae. The patient-specific interbody spinal implant can include a surface morphology based on patient-specific image data of a vertebrae or intervertebral disc (e.g., an intervertebral joint) for which the interbody spinal implant is intended to be implanted and/or normative values of the intervertebral joint or disc. The normative values may be obtained via machine learning techniques. In exemplary embodiments, the surface morphology of the implant may be based on a surgeon's surgical preferences for implantation of the interbody implant, as described herein.

While the subject disclosure has been described with respect to exemplary examples disclosed, those skilled in the art will appreciate that there are numerous variations and permutations of the above-described systems and techniques. It is to be understood that other embodiments may be utilized and structural and functional modifications may be made without departing from the scope of the subject disclosure. Thus, the spirit and scope of the subject disclosure invention should be construed broadly as set forth in the appended claims.

1. A system for generating a patient-specific implant comprising:
a processor configured to:
receive patient-specific image data of a patient's joint;
generate a 3D model of the patient's joint based on the received patient-specific image data;
obtain normative values of joint parameters of individuals other than the patient having patient characteristics similar to the patient, wherein the normative values of the joint parameters of the individuals other than the patient are for a joint type that matches the patient's joint;
modify the generated 3D model of the patient's joint based on the normative values of the joint for the joint type to create a normalized 3D model of the patient's joint; and
generate a 3D model of a joint implant based on the normalized 3D model of the patient's joint.

2. The system of claim 1, wherein the 3D model of the joint implant is an interbody spinal implant, a knee implant, a hip implant, a shoulder implant, an ankle implant, an elbow implant, or a shoulder implant.

3. The system of claim 1, wherein the patient characteristics similar to the patient include at least one of a height, weight, age, sex, activity level, and bone density.

4. The system of claim 1, wherein the patient-specific image data is intra-operative image data of the patient's joint after portions of the joint have been resected.

5. The system of claim 1, wherein the patient characteristics similar to the patient are patient anthropometric characteristics.

6. The system of claim 1, wherein the processor is further configured to modify the normalized 3D model of the patient's joint based on a virtual surgery of the joint.

7. The system of claim 6, wherein the virtual surgery of the joint comprises a virtual resection or milling of a portion of the joint of the normalized 3D model.

8. The system of claim 1, wherein the processor is further configured to:
receive patient-specific medical data of the patient; and
modify the 3D model of the joint implant based on the received patient-specific medical data.

9. The system of claim 1, wherein the obtained normative values are obtained via machine learning techniques trained using data relating to individuals other than the patient.

10. The system of claim 1, wherein the processor is further configured to:
receive surgeon-specific preferences regarding a generic surgery relating to the patient's joint; and
modify the 3D model of the joint implant based on the received surgeon-specific preferences.

11. The system of claim 1, wherein the 3D model of the joint implant includes joint bones and/or fibrocartilage of the joint.

12. A patient-specific interbody spinal implant comprising:
a surface morphology based on patient-specific image data of an intervertebral joint or disc for which the interbody spinal implant is intended to be implanted and normative values of said intervertebral joint or disc of individuals other than the patient and having patient characteristics similar to the patient obtained via machine learning techniques.

13. The patient-specific interbody spinal implant of claim 12, wherein the surface morphology of the implant is further based on a surgeon's surgical preferences for implantation of the interbody implant

14. A method of producing a patient-specific implant comprising:
receiving patient-specific image data of a patient's joint;
creating a 3D model of the patient's joint based on the received patient-specific image data;
obtaining normative values of joint parameters of individuals other than the patient and having patient characteristics similar to the patient, wherein the normative values of the joint parameters of the individuals other than the patient are for a joint type that matches the patient's joint;
modifying the 3D model of the patient's joint based on the obtained normative values of the joint parameters for the joint type to create a normalized 3D model of the patient's joint;
obtaining a 3D model of a joint implant suitable for implantation within the patient's joint; and
modifying the generated 3D model of the joint implant based on the normalized 3D model of the patient's joint.

15. The method of claim 14, further comprising manufacturing a joint implant based on the modified 3D model of the joint implant.

16. The method of claim 14, wherein the 3D model of the joint implant is an interbody spinal implant, a knee implant, a hip implant, a shoulder implant, an ankle implant, an elbow implant, or a shoulder implant.

17. The method of claim 14, wherein the 3D model of the joint implant includes joint bones and/or fibrocartilage of the joint.

18. The method of claim 14, wherein the patient characteristics similar to the patient include at least one of a height, weight, age, sex, activity level, and bone density.

19. The method of claim 14, wherein the method further comprises modifying the normalized 3D model of the patient's joint based on a virtual surgery of the joint.

20. The method of claim 14, wherein the method further comprises:
receiving patient-specific medical data of the patient; and
modifying the 3D model of a joint implant based on the received patient-specific medical data;
receiving surgeon-specific data regarding a surgery for the patient's joint; and
modifying the 3D model of the joint implant based on the received surgeon-specific data.
